(19) United States
(12) Patent Application Publication
(10) Pub. No.: US 2025/0345556 A1
(43) Pub. Date: Nov. 13, 2025

(54) METHOD FOR MANUFACTURING CATHETER

(71) Applicant: TERUMO KABUSHIKI KAISHA, Tokyo (JP)

(72) Inventors: Yuji MOTOSE, Hiratsuka-city (JP); Tsutomu SUGIKI, Fuji-city (JP)

(73) Assignee: TERUMO KABUSHIKI KAISHA, Tokyo (JP)

(21) Appl. No.: 19/278,861

(22) Filed: Jul. 24, 2025

Related U.S. Application Data
(63) Continuation of application No. PCT/JP2024/001586, filed on Jan. 22, 2024.

(30) Foreign Application Priority Data
Jan. 26, 2023 (JP) 2023-010125

(51) Int. Cl. A61M 25/00

(52) U.S. Cl. CPC A61M 25/0009; A61M 2207/00

(57) ABSTRACT

A method for manufacturing a catheter includes, in a position maintaining state in which a plurality of catheter members arranged in a predetermined manner are inserted into a first hollow portion of an elastically deformable elastic body having a laser transmission property and having a first hollow portion having a cross-sectional area smaller than a cross-sectional area of adjacent portions of the plurality of catheter members at no load via an insertion member, and a position of the elastic body is maintained inside a second hollow portion of a hard component having a laser transmission property and harder than the elastic body, irradiating the plurality of catheter members arranged in a predetermined manner via the hard component and the elastic body with a laser beam to fuse the plurality of catheter members arranged in a predetermined manner.

### CROSS-REFERENCES TO RELATED APPLICATIONS

This application is a continuation of International Application No. PCT/JP2024/001586 filed on Jan. 22, 2024, which claims priority to Japanese Application No. 2023-010125 filed on Jan. 26, 2023, the entire content of both of which is incorporated herein by reference.

### TECHNOLOGICAL FIELD

The present disclosure generally relates to a method for manufacturing a catheter.

### BACKGROUND DISCUSSION

When various therapeutic actions are performed in an organ of a living body, a medical instrument including a tubular body constituted by a flexible hollow tubular member is often used. Commonly known instruments of this type of medical instrument include a guiding catheter used for delivering a catheter device such as a balloon catheter to a desired position in a living body, a contrast catheter used for discharging a contrast medium into a living body, and a microcatheter used for discharging a medicine.

The above-described catheter may be manufactured through a joining of hollow tubes by a laser or the like. The above-described conventional technique of joining tubes to each other at a portion where the tubes are to be joined may employ a heat shrinkable tube (see Japanese Patent Application Publication No. 2002-301160 A).

The above-described heat shrinkable tube is disposed so as to cover the catheter members to be processed in a state where these catheter members to be processed are disposed in a predetermined manner, is thermally shrunk in the state to hold the catheter member in a contact state, and is removed from the catheter member after laser processing. However, since the removal operation of the heat shrinkable tube is manually performed by the operator, there is a risk that the catheter member is damaged, and it is necessary to redo the operation when the removal cannot be performed well. In addition, since the heat shrinkable tube shrinks after heating, the heat shrinkable tube cannot be reused and must be discarded after a single use.

As described above, in the method for manufacturing the catheter using the heat shrinkable tube, the influence on the manufacturing cost such as the labor cost required for the removal operation of the heat shrinkable tube and the material cost of the heat shrinkable tube is relatively large, and there is room for improvement.

### SUMMARY

A method is disclosed for manufacturing a catheter capable of saving labor and reducing cost while improving quality at the time of manufacturing the catheter.

(1) A method for manufacturing a catheter including: in a position maintaining state in which a plurality of catheter members arranged in a predetermined manner are inserted into a first hollow portion of an elastically deformable elastic body having a laser transmission property and having a first hollow portion having a cross-sectional area smaller than cross-sectional areas of fusion target portions of the plurality of catheter members at no load via an insertion member, and a position of the elastic body is maintained inside a second hollow portion of a hard component having a laser transmission property and harder than the elastic body, irradiating the plurality of catheter members arranged in a predetermined manner via the hard component and the elastic body with a laser beam to fuse the plurality of catheter members arranged in a predetermined manner.

(2) The method for manufacturing a catheter according to (1), in which an inner peripheral surface of the first hollow portion of the elastic body is configured by a material in which sliding resistance with respect to the catheter member is reduced.

(3) The method for manufacturing a catheter according to (1) or (2), in which cross-sectional shapes of the plurality of catheter members arranged in a predetermined manner are different before and after passing through the first hollow portion of the elastic body.

(4) The method for manufacturing a catheter according to (3), in which cross-sectional areas of the plurality of catheter members arranged in a predetermined manner are reduced by passing through the first hollow portion of the elastic body as compared with a cross-sectional area before passing.

(5) The method for manufacturing a catheter according to (3), in which cross-sectional shapes of the plurality of catheter members arranged in a predetermined manner are shortened in a longitudinal axis (i.e., long axis) direction by passing through the first hollow portion of the elastic body.

(6) The method for manufacturing a catheter according to any one of (1) to (5), in which the plurality of catheter members arranged in a predetermined manner include a first member and a second member arranged in a radial direction.

(7) The method for manufacturing a catheter according to any one of (1) to (5), in which the plurality of catheter members arranged in a predetermined manner include a first member and a second member arranged in an axial direction.

(8) A method for manufacturing a catheter including: in a state in which, in a state where a plurality of catheter members arranged in a predetermined manner on an insertion member positioned coaxially with a first hollow portion is disposed inside the first hollow portion of an elastically deformable elastic body having a laser transmission property and the first hollow portion through which a plurality of catheter members can be inserted at no load, and the elastic body and the plurality of catheter members arranged in a predetermined manner are disposed inside a second hollow portion of a hard component having a laser transmission property and harder than the elastic body, the plurality of catheter members arranged in a predetermined manner and the elastic body are relatively moved in a coaxial direction and a force is applied in a direction in which the hard component reduces an outer diameters of the plurality of catheter members arranged in a predetermined manner via the elastic body, irradiating the plurality of catheter members arranged in a predetermined manner via the hard component and the elastic body with a laser beam to fuse the plurality of catheter members arranged in a predetermined manner.

(9) A method for manufacturing a catheter comprising: inserting a plurality of catheter members via an insertion member into a first hollow portion of an elastically deformable elastic body having a laser transmission property, the first hollow portion having a cross-sectional area smaller than cross-sectional areas of fusion target portions of the plurality of catheter members; maintaining a position of the elastic body inside a second hollow portion of a hard component having a laser transmission property and harder than the elastic body; and irradiating the plurality of catheter members and the elastic body arranged in the predetermined manner via the hard component and the elastic body with a laser beam to fuse the plurality of catheter members arranged in the predetermined manner.

According to the present disclosure, it is possible to reduce labor and cost while improving quality of a catheter manufactured by fusing a plurality of catheter members.

### BRIEF DESCRIPTION OF THE DRAWINGS

FIG. 1 is a schematic configuration diagram of a catheter manufactured by a method for manufacturing a catheter according to the present disclosure.

FIG. 2 is a view schematically illustrating a manufacturing device used in the method for manufacturing the catheter according to the embodiment.

FIG. 3 is a cross-sectional view of a hard component included in the manufacturing device according to the embodiment.

FIG. 4 is a flowchart illustrating the method for manufacturing the catheter according to the embodiment.

FIG. 5A is a view for explaining the method for manufacturing the catheter according to the embodiment.

FIG. 5B is a view for explaining the method for manufacturing the catheter according to the embodiment.

FIG. 5C is a view for explaining the method for manufacturing the catheter according to the embodiment.

FIG. 5D is a view for explaining the method for manufacturing the catheter according to the embodiment.

FIG. 6 is an enlarged view of a broken line portion 6A illustrated in FIG. 5D.

FIG. 7A is a view illustrating an elastic body according to a first modification.

FIG. 7B is a view illustrating an elastic body according to a second modification.

FIG. 7C is a view illustrating an elastic body according to a third modification.

FIG. 8A is a view illustrating a method for manufacturing a catheter according to a first configuration example.

FIG. 8B is a view illustrating a part of the catheter according to the first configuration example.

FIG. 9A is a view illustrating a method for manufacturing a catheter according to a second configuration example.

FIG. 9B is a view illustrating a part of a catheter according to a second configuration example.

FIG. 10A is a view illustrating a method for manufacturing a catheter according to a third configuration example.

FIG. 10B is an axis-orthogonal cross-sectional view illustrating a part of the catheter according to the third configuration example.

FIG. 10C is an axis-orthogonal cross-sectional view illustrating a part of the catheter according to the third configuration example.

### DETAILED DESCRIPTION

Hereinafter, a mode for carrying out the present disclosure will be described in detail with reference to the drawings. Embodiments herein described are illustrated to embody the technical idea of the present disclosure and do not limit the present disclosure. Other feasible embodiments, examples, operation technologies and the like that could be conceived by those skilled in the art without departing from the gist of the present disclosure are all included in the scope and gist of the present disclosure and included in the disclosure recited in claims and the scope of equivalents thereof.

Moreover, for convenience of illustration and ease of understanding, the drawings attached to the present specification may be schematically represented by changing a scale, an aspect ratio, a shape, and the like, from actual ones as appropriate, but are merely examples, and do not limit the interpretation of the present disclosure.

In the present specification, the following directions are defined for convenience of description. The “axial direction” of a catheter body 10 and a catheter member 30 means a longitudinal direction in which the catheter body 10 and the catheter member 30 extend, and is a direction along the “central axis C” illustrated in the drawing. In the present specification, a direction along the same direction as the above-described “axial direction” is also referred to as a “coaxial direction”.

In addition, the “radial direction” of the catheter body 10 and the catheter member 30 is a direction vertically away from or approaching the central axis C. The “circumferential direction” of the catheter body 10 and the catheter member 30 is a direction of a set of points separated by a predetermined distance with respect to the central axis C.

Note that, in the following description, ordinal numerals such as “first” and “second” will be given, but are used for convenience and do not define any order unless otherwise specified.

A catheter 100 manufactured by the method for manufacturing a catheter according to the present embodiment may be inserted into a blood vessel, a bile duct, a trachea, an esophagus, a urethra, or other cavities or lumens in a living body to be used for treatment, diagnosis, or the like. The catheter 100 can be, for example, a balloon catheter, a microcatheter, a contrast catheter, a guiding catheter, or the like for percutaneous transluminal coronary angioplasty (PTCA) or percutaneous transluminal angioplasty (PTA). In addition, a device described in a configuration example described later can be appropriately used.

### Catheter

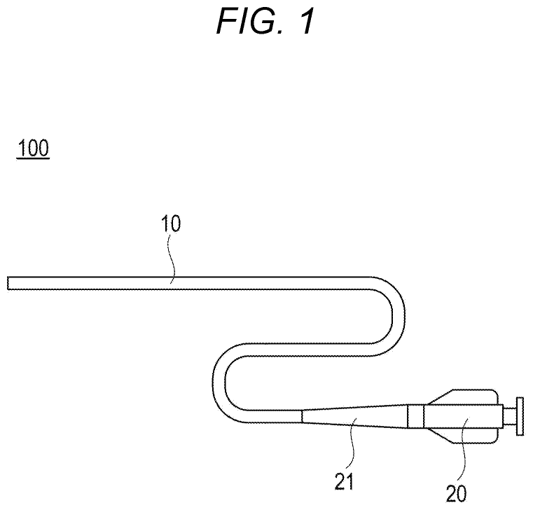

FIG. 1 is a schematic diagram illustrating the catheter 100 manufactured by the method for manufacturing a catheter according to an embodiment of the present disclosure.

As illustrated in FIG. 1, the catheter 100 is configured to provide an elongated catheter body 10 that can be introduced into a living body, a hub 20 connected to a proximal end portion of the catheter body 10, and an anti-kink protector 21 in the vicinity of a connecting portion between the catheter body 10 and the hub 20. Note that the specific configuration of the catheter 100 is not particularly limited, and for example, the anti-kink protector 21 may not be provided.

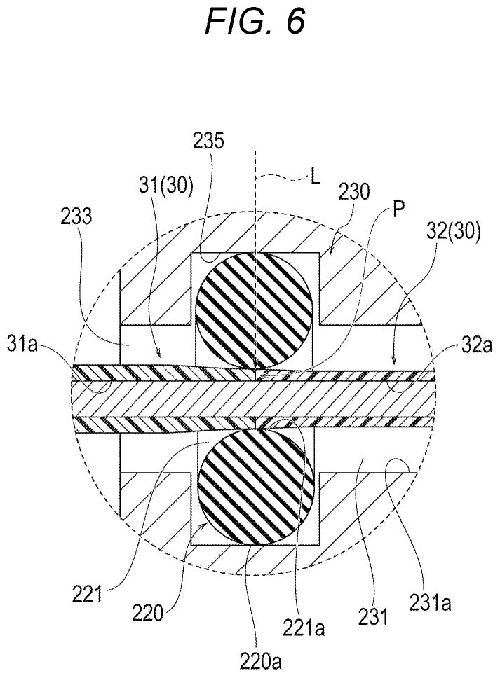

The catheter body 10 is configured as a flexible tubular member in which a lumen extending in the axial direction is formed. As will be described later in the present embodiment, the catheter body 10 is formed by fusing an adjacent portion (corresponding to a “fusion target portion”) P where the end portion of a first member 31 and the end portion of a second member 32 are abutted and brought into contact with each other in a state where two cylindrical members of the first member 31 and the second member 32 are arranged in the axial direction as the catheter member 30 (see FIG. 6). The adjacent portion P is provided at one end portion of the first member 31 and the second member 32 in the axial direction.

Examples of the constituent material of the first member 31 and the second member 32 can include, in addition to a polyamide resin, a polyester resin, a polyolefin resin, and a polyurethane resin, a polyamide elastomer, a polyester elastomer, a polyurethane elastomer, or a mixture of one or more of the above-mentioned constituent materials of the first member 31 and the second member 32 or a mixture of two or more of the above-mentioned constituent materials of the first member 31 and the second member 32 having different hardness. Three or more members configured by various resins or elastomers having different hardness may be arranged so as to be flexible from the proximal end toward the distal end. In addition, in order to harden a predetermined portion and soften the other portion, members configured by resin or elastomer having hardness corresponding to the portion may be arranged. These members of resin or elastomer and arranged by corresponding hardness can be used as, for example, a first tube to be an outer layer in a second configuration example to be described later after fusion bonding. Furthermore, in at least one of the first member 31 and the second member 32, a resin layer having higher slidability than the resin exemplified above, for example, a layer configured by polytetrafluoroethylene resin may be provided inside the tubular member (cylindrical member) in order to enhance the slidability of the inner surface.

The constituent materials of the first member 31 and the second member 32 preferably include the same kind of constituent materials. As an example, a constituent material of the first member 31 is a polyamide elastomer, and a constituent material of the second member 32 is a polyamide resin. However, the first member 31 and the second member 32 may be configured by materials different from the above materials. As an example, the constituent material of the first member 31 may be a polyester elastomer, and the constituent material of the second member 32 may be a polyester resin. In addition, as long as a predetermined bonding strength or more can be obtained as described above, the first member 31 and the second member 32 may be configured by different materials.

In addition, the first member 31 and the second member 32 can contain a pigment or dye that develops white, black, blue, red, yellow, or the like, and a mixture of the pigment or dye that develops white, black, blue, red and/or yellow. Such a pigment or dye may be selected from materials that absorb a laser beam L and generate heat. Examples of the material of the pigment or dye that generates heat by absorbing a laser beam L can include, for example, carbon black.

The first member 31 and the second member 32 can be configured to include a powdered X-ray contrast material. Specific examples of the powdered X-ray contrast material can include, for example, compounds of gold, titanium, bismuth, and tungsten. Furthermore, in the first member 31 and the second member 32, a reinforcing body configured by tungsten, SUS (i.e., stainless steel), or the like may be disposed in the above-described material. As the reinforcing body, for example, a coiled or blade-shaped reinforcing body can be used.

The hub 20 is liquid-tightly fixed to the catheter body 10 with an adhesive, a fixing tool, or the like. The hub 20 functions as a port through which a guide wire is inserted into the lumen of the catheter body 10, or an injection port through which a liquid medicine, an embolic substance, a contrast medium, or the like is injected into the lumen, and also functions as a grip portion when the catheter 100 is operated. Examples of a material usable for the hub 20 can include a thermoplastic resin such as polycarbonate, polyamide, polysulfone, or polyarylate.

Note that, when the catheter 100 has the anti-kink protector 21 as illustrated in FIG. 1, the anti-kink protector 21 can be configured by an elastic material provided so as to surround a part of the proximal end portion of the catheter body 10. As a constituent material of the anti-kink protector 21, natural rubber, silicone resin, or the like can be used, for example.

### Manufacturing Device

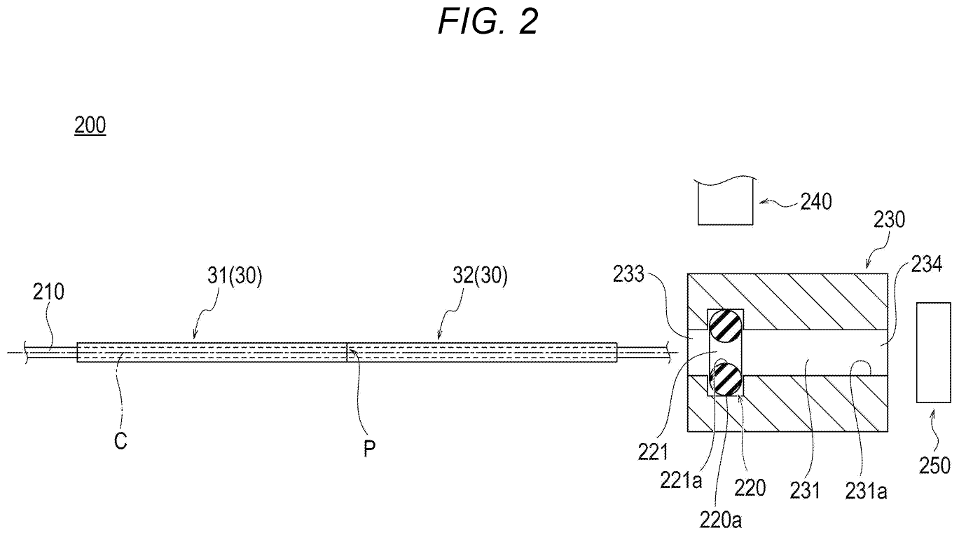

Next, a catheter manufacturing device 200 (hereinafter, also simply referred to as “manufacturing device 200”) according to an embodiment will be described. FIG. 2 is a schematic configuration diagram of the manufacturing device 200.

The manufacturing device 200 includes an insertion member 210, an elastic body 220, a hard component 230, a laser irradiation unit 240, and a holding mechanism 250.

### Insertion Member

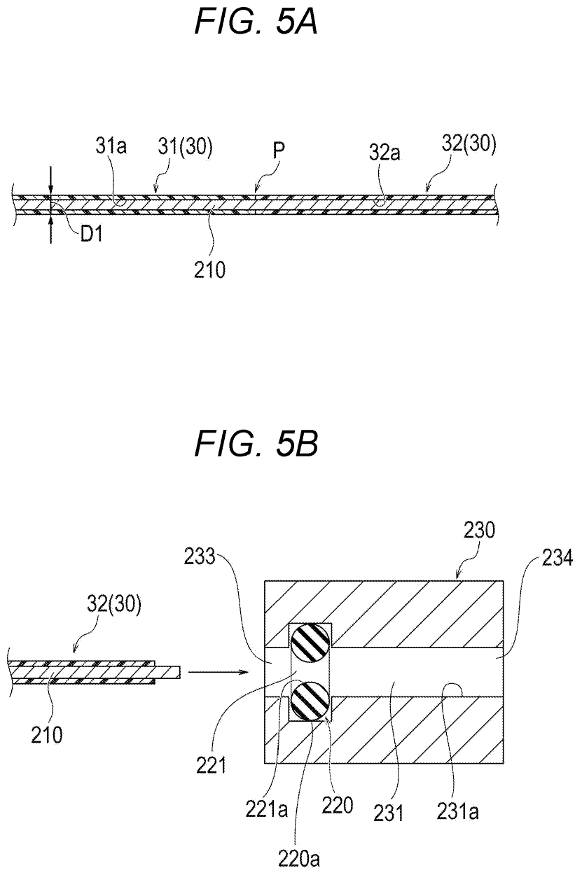

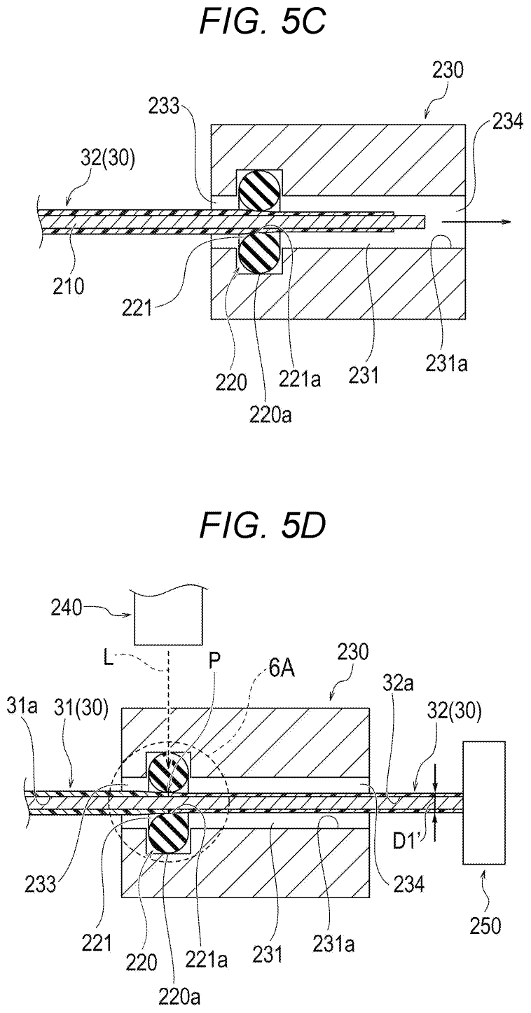

The insertion member 210 is configured to be insertable through a lumen 31a of the cylindrical first member 31 and a lumen 32a of the second member 32 to be fused (see FIG. 5A).

The insertion member 210 can be configured by, for example, a core metal. The insertion member 210 can be configured by a metal material or the like having heat resistance when the first member 31 and the second member 32 are fused and rigidity capable of holding the positions of the members 31 and 32.

The insertion member 210 can be configured by a cylindrical member having a circular cross-sectional shape intersecting the axial direction. However, the cross-sectional shape, the material, the length, the outer diameter, and the like of the insertion member 210 are not particularly limited as long as the insertion member has a configuration in which the first member 31 and the second member 32 can be inserted. The insertion member 210 may be configured by, for example, a hollow tubular member.

### Elastic Body

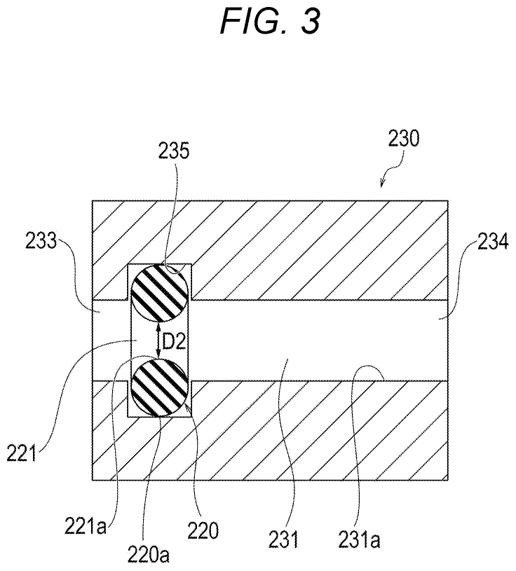

As illustrated in FIGS. 3, 5C, and 5D, the elastic body 220 includes a first hollow portion 221 having a cross-sectional area smaller than the cross-sectional area of the fusion target portion of the first member 31 and the second member 32 attached to the insertion member 210.

As illustrated in FIGS. 5D and 6, the elastic body 220 is configured to be elastically deformable so as to be able to apply a force inward in the radial direction to the adjacent portion P between the first member 31 and the second member 32. The elastic body 220 is configured to maintain a predetermined annular shape as illustrated in FIGS. 3 and 5B before and after the first member 31 and the second member 32 are inserted into the first hollow portion 221.

The elastic body 220 can be disposed, for example, near the inlet portion 233 of the second hollow portion 231 of the hard component 230. The outer peripheral surface 220a of the elastic body 220 is disposed so as to maintain a state of being in contact with a predetermined position of the inner peripheral surface 231a of the second hollow portion 231 of the hard component 230. The elastic body 220 can be disposed so as not to cause positional displacement by being fixed to the hard component 230, for example, by being fitted into a recessed groove portion provided inside the second hollow portion 231 of the hard component 230. In the embodiment of FIG. 3, the predetermined position of the inner peripheral surface 231a of the second hollow portion 231 of the hard component 230 is an inner surface portion 235 of the recessed groove portion.

The cross-sectional area of the first hollow portion 221 of the elastic body 220 (dimension corresponding to the inner diameter D2 illustrated in FIG. 3) is smaller than the cross-sectional area of each of the members 31 and 32 (dimension corresponding to the outer diameter D1 illustrated in FIG. 5A). Therefore, as illustrated in FIGS. 5C, 5D, and 6, when the first member 31 and the second member 32 are inserted into the first hollow portion 221, at least a part of the inner peripheral surface 221a of the first hollow portion 221 is brought into pressure contact with the outer surface of each of the members 31 and 32. In this state, since the outer peripheral surface 220a of the elastic body 220 is restrained by the inner peripheral surface 231a of the hard component 230, the deformation of the elastic body 220 so as to spread outward in the radial direction is restricted. As a result, the elastic body 220 can apply a pressing force to the members 31 and 32 located on the inner peripheral surface 221a side of the first hollow portion 221. When the adjacent portion P is disposed inside the first hollow portion 221, the elastic body 220 holds both the members 31 and 32 in a state of abutting each other at the adjacent portion P and the peripheral portion of the adjacent portion P. In the present specification, a state in which the elastic body 220 applies an external force to the adjacent portion P, which is the fusion target portion of the catheter member 30, in a state in which the position of the elastic body 220 is maintained inside the second hollow portion 231 of the hard component 230 is referred to as a “position maintaining state”.

In addition, in a state where the plurality of catheter members 30 arranged in a predetermined manner on the insertion member 210 located coaxially with the first hollow portion 221 of the elastic body 220 is disposed inside the first hollow portion 221, and the elastic body 220 and the plurality of catheter members 30 arranged in a predetermined manner are disposed inside the second hollow portion 231 of the hard component 230, the catheter member 30 and the elastic body 220 are relatively moved in the coaxial direction, whereby the elastic body 220 can apply an external force to the adjacent portion P which is the fusion target portion of the catheter member 30.

In the above-described position maintaining state, the hard component 230 applies a force in a direction of reducing the outer diameters of the plurality of catheter members 30 via the inner peripheral surface 221a of the first hollow portion 221 of the elastic body 220. By irradiating the adjacent portion P with the laser beam L in the above-described position maintaining state, the members 31 and 32 are appropriately fused to each other in a state where an external force is applied to the adjacent portion P.

When the catheter member 30 and the elastic body 220 are moved coaxially, the elastic body 220 is fixed and the catheter member 30 is moved in the above description, but the catheter member 30 may be fixed and the elastic body 220 may be moved. In addition, both the catheter member 30 and the elastic body 220 may be moved in directions facing each other.

The elastic body 220 can be configured to include a material having a laser transmission property that enables the adjacent portion P to be fused when the laser beam L is emitted from the outside of the hard component 230 toward the adjacent portion P disposed inside the first hollow portion 221 and the second hollow portion 231.

In the present specification, the “laser transmission property” means that the elastic body 220 contains a material having a transmittance of 80% or more with respect to the laser beam L per 1 mm of the thickness of the elastic body in the radial direction.

The inner peripheral surface 221a of the first hollow portion 221 of the elastic body 220 can be configured by, for example, a material having reduced sliding resistance with respect to the first member 31 and the second member 32 inserted into the first hollow portion 221 of the elastic body 220. In addition, the elastic body 220 preferably contains a material having higher heat resistance than the first member 31 and the second member 32. As a material suitable for the above-described purpose (i.e., a material for reducing sliding resistance with respect to the first member 31 and the second member 32 inserted into the first hollow portion 221 of the elastic body 220) can be, for example, silicone rubber, fluororubber, or the like.

### Hard Component

The hard component 230 has a laser transmission property. In addition, the hard component 230 can be configured by a member harder than the elastic body 220.

Similar to the elastic body 220, the laser transmission property of the hard component 230 means that the hard component 230 is configured to have transmittance, for example, of 80% per 1 mm of the thickness of the elastic body in the radial direction. As a constituent material of the hard component 230, can be, for example, a material having a laser transmission property and harder than the elastic body 220. Furthermore, it is more preferable to use a material that hardly deforms due to heat transfer from a workpiece that generates heat by irradiation with the laser beam L. As such a material, quartz glass or the like can be suitably used.

As illustrated in FIG. 3, the hard component 230 has a second hollow portion 231 in which the elastic body 220 is fixedly disposed. When the adjacent portion P is fused, the members 31 and 32 are moved into the first hollow portion 221 of the elastic body 220, so that the members 31 and 32 can be disposed in the second hollow portion 231 of the hard component 230.

As illustrated in FIGS. 3, 5B, and 5C, the second hollow portion 231 of the hard component 230 has an inlet portion 233 and an outlet portion 234 positioned in the moving direction (hereinafter, also simply referred to as “moving direction”) of the members 31 and 32 arranged in the insertion member 210.

The members 31 and 32 can be inserted into the second hollow portion 231 via the inlet portion 233. A part or the whole of the members 31 and 32 can be taken out to the outside of the second hollow portion 231 via the outlet portion 234 located on the opposite side of the inlet portion 233. In the present embodiment, as illustrated in FIGS. 5D and 6, when the members 31 and 32 are fused, a predetermined range in the axial direction including the adjacent portions P of the members 31 and 32 is disposed inside the first hollow portion 221 and the second hollow portion 231.

The shapes of the elastic body 220 (the outer shape and the cross-sectional shape of the first hollow portion 221) and the shapes of the hard component 230 (the outer shape and the cross-sectional shape of the first hollow portion 221) are not particularly limited as long as the elastic body 220 can be disposed inside the second hollow portion 231, the hard component 230 can be disposed radially outside the elastic body 220, the members 31 and 32 can be inserted into the first hollow portion 221 to maintain the positions, and the shapes correspond to the final shapes of the members 31 and 32 after fusion. For example, the cross-sectional shape of the first hollow portion 221 of the elastic body 220 can be any shape according to the cross-sectional shape of the elastic body 220. Furthermore, for example, the cross-sectional shape of the second hollow portion 231 of the hard component 230 can be any shape according to the cross-sectional shape of the elastic body 220.

### Laser Irradiation Unit

The laser irradiation unit 240 is used when the adjacent portion P and the like are fused via the elastic body 220 and the hard component 230. The laser irradiation unit 240 includes a light source that oscillates a laser beam L, a galvano scan (scanner) that changes the laser beam L oscillated from the light source in a predetermined direction by a motor, a mirror, or the like, a prism, and the like. One or a plurality of the laser irradiation units 240 are disposed at positions (around the hard component 230) where the adjacent portion P to be the fusion target portion of the catheter member 30 can be irradiated with the laser beam L.

The laser irradiation unit 240 emits the laser beam L having a wavelength that causes a fusion target portion such as the adjacent portion P to generate heat by radiation heating. The spot diameter of the laser beam L can be set to 0.1 mm to @10 mm, and the wavelength of the laser beam L can be set to 800 nm to 10,000 nm. When the laser beam L is emitted toward the adjacent portion P, the laser beam L passes through the hard component 230 and the elastic body 220, and forms a fusion bonded portion in the adjacent portion P.

As the fusion target portion irradiated with the laser beam L, a portion to be processed in the catheter member 30 can be arbitrarily selected. For example, in a state where a plurality of members to be the catheter member 30 are disposed side by side in the axial direction or the radial direction, end surfaces of the members and a peripheral portion of the members, a portion overlapping each other in a state where the two members are partially overlapped, a peripheral portion of the members, and the like can be set as the fusion target region.

### Holding Mechanism

As illustrated in FIGS. 2 and 5D, the manufacturing device 200 includes a holding mechanism 250 for controlling axial movement and/or circumferential movement (rotation) of the first member 31 and the second member 32 when processing the catheter member 30.

The holding mechanism 250 can be configured by, for example, a chuck (bearing jig). The manufacturing device 200 can be configured to control operation of the holding mechanism 250 via a motor and a gear. By causing the holding mechanism 250 to hold the end portion of the catheter member 30 and the end portion of the insertion member 210, these members can be set in the manufacturing device 200 in a state of being movable in the axial direction and the circumferential direction. The movement of the members 31 and 32 into the first hollow portion 221 of the elastic body 220, the movement of the adjacent portion P of the catheter member 30 after fusion, and the removal of the members 31 and 32 from the second hollow portion 231 of the hard component 230 can be appropriately performed by the operation of the holding mechanism 250.

In the example illustrated in FIG. 2, the manufacturing device 200 includes one holding mechanism 250 disposed on the outlet portion 234 side of the hard component 230. However, in the manufacturing device 200, the holding mechanism 250 may be disposed on the inlet portion 233 side of the hard component 230. In the manufacturing device 200, the holding mechanism 250 may be disposed on the outlet portion 234 side and the inlet portion 233 side. The holding mechanism 250 on the inlet portion 233 side and the outlet portion 234 side can also be controlled to synchronize the axial movement and/or the circumferential movement (rotation).

### Method for Manufacturing Catheters

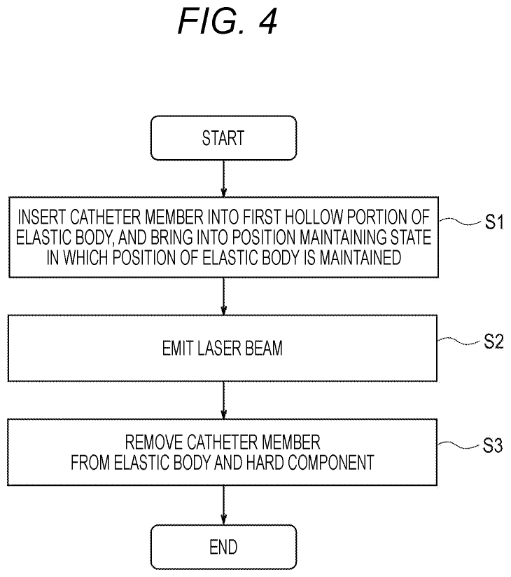

Next, a method for manufacturing a catheter according to the present embodiment will be described. FIG. 4 is a flowchart illustrating a method for manufacturing the catheter 100, and FIGS. 5A to 5D are views schematically illustrating each step of the method for manufacturing the catheter including a preparation stage based on the flowchart illustrated in FIG. 4.

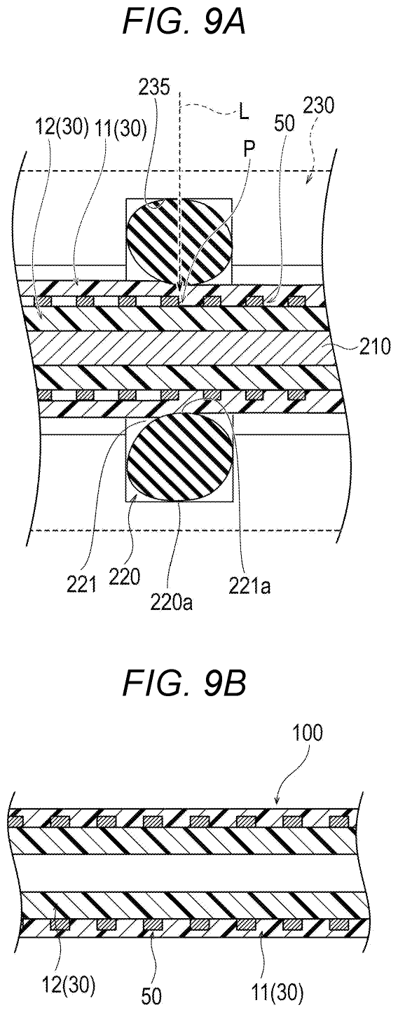

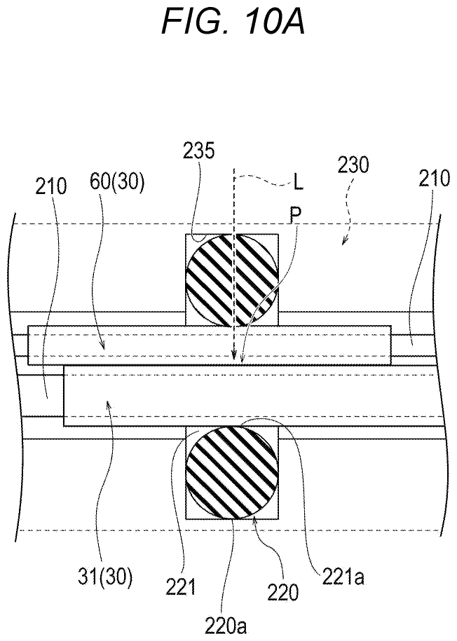

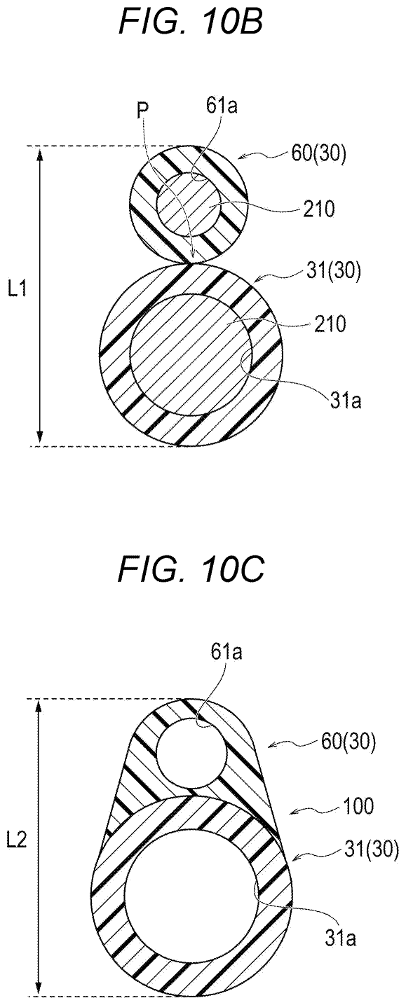

As illustrated in FIG. 5A, the plurality of catheter members 30 (the first member 31 and the second member 32) arranged in a predetermined manner via the insertion member 210 is prepared. Note that, in the present specification, “arranged in a predetermined manner via the insertion member 210” includes not only a state in which the members 31 and 32 are coaxially disposed via one insertion member 210 as shown in the present embodiment, but also a state in which a relative positional relationship between the members 31 and 32 inserted into one or a plurality of insertion members 210 is arranged at different positions in a radial direction or an axial direction with respect to a position of the insertion member 210 as shown in a configuration example and the like to be described later (see FIGS. 9A and 10A).

As illustrated in FIGS. 5B and 5C, the plurality of catheter members 30 arranged in a predetermined manner via the insertion member 210 are moved into the first hollow portion 221 of the elastic body 220. When the plurality of catheter members 30 are moved into the first hollow portion 221 of the elastic body 220, the plurality of catheter members 30 are moved into the second hollow portion 231 of the hard component 230.

As illustrated in FIG. 5D, after a part of the catheter member 30 is taken out from the outlet portion 234 side of the hard component 230, the end portion of the catheter member 30 is held by the holding mechanism 250. When the catheter member 30 is held by the holding mechanism 250, it is possible to control the axial movement and/or rotation operation using the holding mechanism 250.

As illustrated in FIG. 5D, the plurality of catheter members 30 arranged in a predetermined manner via the insertion member 210 are moved inside the first hollow portion 221 of the elastic body 220 (S1 in FIG. 4). By moving the plurality of catheter members 30 into the first hollow portion 221 of the elastic body 220, the plurality of catheter members 30 can be moved into the second hollow portion 231 of the hard component 230. As a result, in a state where the position of the elastic body 220 is maintained inside the second hollow portion 231 of the hard component 230, a position maintaining state in which the elastic body 220 applies an external force to the adjacent portion P to be fused in the catheter member 30 can be achieved.

In the present embodiment, the inner peripheral surface 221a of the first hollow portion 221 of the elastic body 220 is configured by a material having reduced sliding resistance with respect to the first member 31 and the second member 32 inserted into the first hollow portion 221. Therefore, the members 31 and 32 can be smoothly inserted into the first hollow portion 221 of the elastic body 220.

In addition, the cross-sectional area (inner diameter D2) of the first hollow portion 221 of the elastic body 220 is smaller than the cross-sectional area (outer diameter D1) of the catheter member 30 (first member 31, second member 32). Therefore, by inserting the members 31 and 32 into the first hollow portion 221 of the elastic body 220, it is possible to form predetermined shapes such that the cross-sectional shapes of the members 31 and 32 are different before and after the insertion of the elastic body 220. In the present embodiment, as described above, since the cross-sectional area (inner diameter D2) of the elastic body 220 is smaller than the cross-sectional area (outer diameter D1) of each of the members 31 and 32, the cross-sectional areas of the members 31 and 32 are smaller after passing through the elastic body 220 than before passing through the elastic body.

As illustrated in FIGS. 5D and 6, the laser irradiation unit 240 is operated to irradiate the adjacent portion P of the catheter member 30 with the laser beam L (S2 in FIG. 4). Thus, a fusion bonded portion is formed from the outer periphery to the inside of the adjacent portion P between the first member 31 and the second member 32 by heat generation of the first member 31 and the second member 32 and/or heat transfer from the insertion member 210 to the first member 31 and the second member 32. Although it has been described that the timing of the irradiation of the laser beam L can be, for example, after reaching the external force application state as described above, it is also possible to substantially simultaneously perform the irradiation of the laser beam L and the change to the external force application state by adjusting the moving speed in the axial direction of the holding mechanism 250 and the output of the laser beam L.

In a case where the adjacent portion P is fused along the circumferential direction, the adjacent portion P is irradiated with the laser beam L while the holding mechanism 250 operates to rotate the catheter member 30 and the hard component 230. In addition, in a case where the fusion bonded portion is formed over a predetermined range in the axial direction of the catheter member 30, the laser beam L is emitted to the catheter member 30 while the holding mechanism 250 operates to move the catheter member 30 in the axial direction.

The operation of the holding mechanism 250 and the irradiation timing of the laser beam L can be controlled on the basis of a procedure and output stored in advance by the control unit. The operation of the holding mechanism 250 and the irradiation timing of the laser beam L may be manually performed.

In the method for manufacturing a catheter according to the present embodiment, when the catheter member 30 is inserted into the first hollow portion 221, the elastic body 220 receives an external force from the inner peripheral surface 231a of the second hollow portion 231 of the hard component 230 and enters a position maintaining state. Therefore, for example, in a case where the fusion target portion is set over a predetermined range in the axial direction of the catheter member 30, or in a case where the fusion target portion is set at a plurality of different positions in the axial direction of the catheter member 30, by moving the catheter member 30 in the axial direction or stopping the movement as necessary, the irradiation of the predetermined position of the catheter member 30 with the laser beam L can be intermittently or continuously performed. Therefore, it is not necessary to set the catheter member 30 in a positioning jig, a dedicated mold, or the like of the manufacturing device every time the fusion bonded portion is formed in the catheter member 30, and thus, it is possible to greatly improve the work efficiency of the manufacturing operation of the catheter 100.

After the fusion of the adjacent portion P is completed, the catheter member 30 is removed from the elastic body 220 and the hard component 230 (S3 in FIG. 4). In the present embodiment, the position maintaining state can be released by moving the catheter member 30 in the axial direction toward the outlet portion 234 side of the hard component 230 and moving the catheter member 30 to the outside of the hard component 230.

After the catheter member 30 is removed from the hard component 230, the manufacturing of the catheter 100 illustrated in FIG. 1 can be completed by performing an operation of attaching the anti-kink protector 21 and the hub 20.

As described above, the method for manufacturing a catheter according to the present embodiment includes, in a position maintaining state in which a plurality of catheter members 30 arranged in a predetermined manner are inserted into a first hollow portion 221 of an elastically deformable elastic body 220 having a laser transmission property and having a first hollow portion 221 having a cross-sectional area smaller than cross-sectional areas of fusion target regions (adjacent portions P) of the plurality of catheter members 30 at no load via an insertion member 210, and a position of the elastic body 220 is maintained inside a second hollow portion 231 of a hard component 230 having a laser transmission property and harder than the elastic body 220, irradiating the plurality of catheter members 30 arranged in a predetermined manner via the hard component 230 and the elastic body 220 with a laser beam L to fuse the plurality of catheter members 30 arranged in a predetermined manner.

According to the above-described method for manufacturing a catheter, in a position maintaining state in which, when the laser beam L is emitted, in a state in which the position of the elastic body 220 is maintained inside the second hollow portion 231 of the hard component 230, the elastic body 220 applies an external force to the adjacent portion P of the catheter member 30, a fusion bonded portion can be formed in the catheter member 30 by emitting the laser beam L via the elastic body 220 and the hard component 230. Therefore, in the case of fusion using a heat shrinkable tube, it is necessary to remove the heat shrinkable tube from the catheter member 30 after fusion, but such removal work becomes unnecessary by using the method according to the present embodiment. When the position maintaining state is released, the catheter member 30 in which the fusion bonded portion is formed can be easily taken out from the first hollow portion 221 of the elastic body 220. Therefore, the elastic body 220 can be used a plurality of times by being reused unlike a heat shrinkable tube used each time. Therefore, in the above-described method for manufacturing a catheter, the risk of damaging the catheter member 30 is extremely small as compared with a conventional method for manufacturing a catheter using a heat shrinkable tube, and labor saving and cost reduction can be achieved while improving quality at the time of manufacturing the catheter 100.

In addition, according to the above-described method for manufacturing a catheter, by moving the catheter members 30 arranged in a predetermined manner into the first hollow portion 221 of the elastic body 220, the position maintaining state, which is a preparation stage before forming the fusion bonded portion by irradiation with the laser beam L, can be achieved. Furthermore, according to the above-described method for manufacturing a catheter, after the fusion bonded portion is formed, the position maintaining state can be released by moving the catheter members 30 arranged in a predetermined manner to the outside of the first hollow portion 221 of the elastic body 220. Therefore, in the above-described method for manufacturing a catheter, it is not necessary to add a complicated manufacturing process for bringing the catheter into a position maintaining state or to add a complicated mechanism to the manufacturing device, as compared with the existing manufacturing method. Therefore, it is possible to achieve further labor saving and cost reduction when manufacturing the catheter 100.

In addition, the method for manufacturing the catheter according to the present embodiment includes: in a state in which, in a state where a plurality of catheter members 30 arranged in a predetermined manner on an insertion member 210 positioned coaxially with a first hollow portion 221 is disposed inside the first hollow portion 221 of an elastically deformable elastic body 220 having a laser transmission property and the first hollow portion 221 through which a plurality of catheter members 30 can be inserted at no load, and the elastic body 220 and the plurality of catheter members 30 arranged in a predetermined manner are disposed inside a second hollow portion 231 of a hard component 230 having a laser transmission property and harder than the elastic body 220, the plurality of catheter members 30 arranged in a predetermined manner and the elastic body 220 are relatively moved in a coaxial direction, irradiating the plurality of catheter members 30 arranged in a predetermined manner via the hard component 230 and the elastic body 220 with a laser beam L to fuse the plurality of catheter members 30 arranged in a predetermined manner.

According to the above-described method for manufacturing a catheter, in a state in which, in a state where the elastic body 220 and the plurality of catheter members 30 arranged in a predetermined manner are disposed inside the second hollow portion 231 of the hard component 230, the plurality of catheter members 30 arranged in a predetermined manner and the elastic body 220 are relatively moved in the coaxial direction, a fusion bonded portion can be formed in the catheter member 30 by emitting the laser beam L via the hard component 230 and the elastic body 220. Accordingly, in the above-described method for manufacturing a catheter, the risk of damaging the catheter member 30 is extremely small as compared with a conventional method for manufacturing a catheter using a heat shrinkable tube, and labor saving and cost reduction can be achieved while improving quality at the time of manufacturing the catheter 100.

### Modification of Elastic Body

The specific configuration used in the method for manufacturing a catheter according to the present disclosure is not particularly limited as long as the elastic body 220 can be brought into the position maintaining state by moving the catheter member 30 into the first hollow portion 221 of the elastic body 220. Hereinafter, the first to third modifications of the elastic body 220 described in the above-described embodiment will be exemplified.

### First Modification of Elastic Body

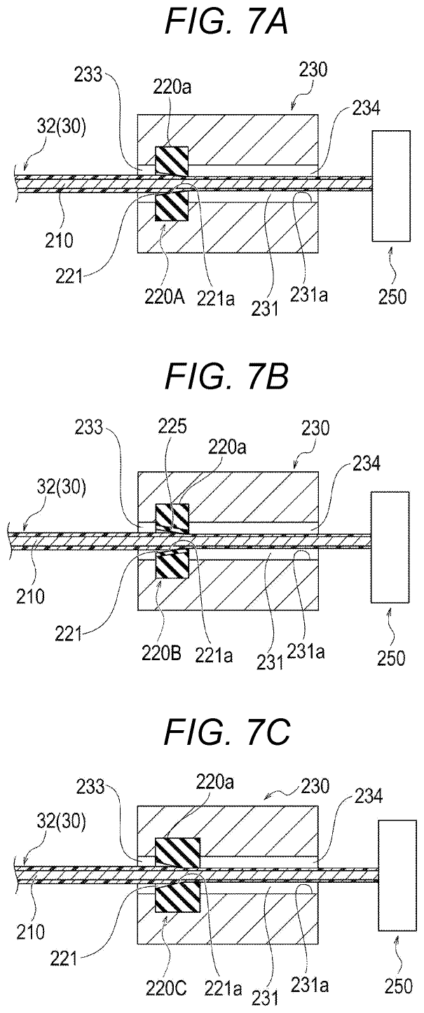

As illustrated in FIG. 7A, the first hollow portion 221 of an elastic body 220A can be configured to have, for example, a tapered cross section in which the cross-sectional area gradually decreases from the end portion side located on the inlet portion 233 side of the hard component 230 toward the end portion side located on the outlet portion 234 side of the hard component 230. With such a configuration, the catheter member 30 is smoothly inserted into the first hollow portion 221 of the elastic body 220A.

### Second Modification of Elastic Body

As illustrated in FIG. 7B, a coating layer 225 for reducing sliding resistance to the catheter member 30 can be disposed on the inner peripheral surface 221a of the first hollow portion 221 of an elastic body 220B. In the case of such a configuration, it is possible to smoothly insert the catheter member 30 into the first hollow portion 221 of the elastic body 220B and move the catheter member 30 inside the first hollow portion 221. The constituent material of the coating layer 225 is not particularly limited, but for example, it can be configured by a known material such as fluororubber or silicone rubber.

### Third Modification of Elastic Body

As illustrated in FIG. 7C, the first hollow portion 221 of an elastic body 220C can include, for example, a tapered portion whose cross-sectional area gradually decreases from the end portion side located on the inlet portion 233 side of the hard component 230 to a predetermined position in the moving direction, and a straight-shaped portion whose cross-sectional area extending rearward in the traveling direction from the tapered portion is substantially constant. In the case of such a configuration, the catheter member 30 can be smoothly inserted into the first hollow portion 221 of the elastic body 220C.

### Configuration Example of Catheter Member

In the method for manufacturing a catheter according to the above-described embodiment, the two parts of the first member 31 and the second member 32 to be the catheter members 30 are arranged in the axial direction, and the adjacent portions P of the two parts are fused by the laser beam L. However, in the method for manufacturing a catheter of the embodiment, the catheter member 30 can be configured as each configuration example described below.

In the first to third configuration examples, the catheter member 30 has a configuration in which a part of the catheter 100 or the catheter 100 and other members are disposed in a predetermined manner. Note that, in some of the views for explaining each configuration example, the hard component 230 included in the manufacturing device 200 is simply illustrated using an alternate long and short dash line.

### First Configuration Example

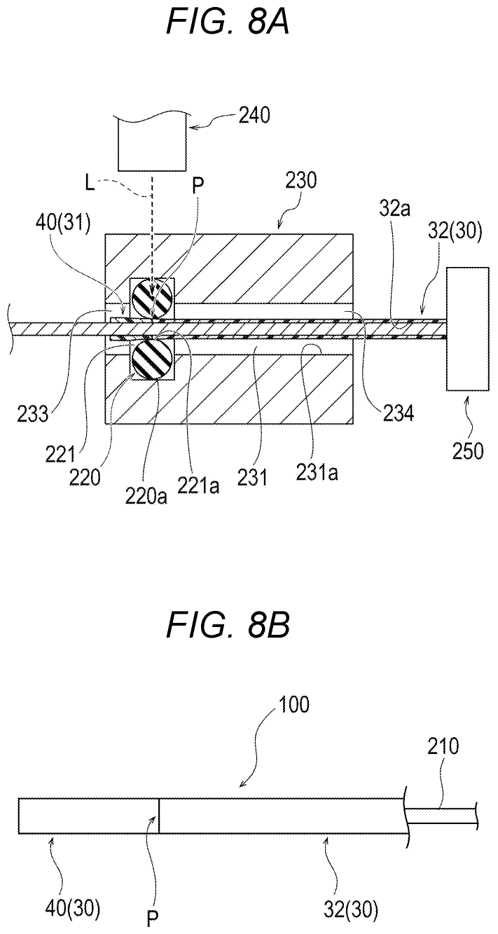

As illustrated in FIGS. 8A and 8B, the catheter member 30 of the first configuration example includes a distal end tip 40 (corresponding to a “first member”) connected to a distal end of the catheter body 10, and a second member 32 constituting the catheter body 10.

When the catheter member 30 of the first configuration example is fused, as illustrated in FIG. 8A, the insertion member 210 is inserted through the distal end tip 40 and the second member 32. The distal end tip 40 through which the insertion member 210 is inserted and the second member 32 are moved to the inside of the first hollow portion 221 of the elastic body 220 of the manufacturing device 200 described above, so that the position is maintained. Then, the laser beam L is emitted toward the adjacent portion P between the distal end tip 40 and the second member 32 to form a fusion bonded portion.

In the method for manufacturing a catheter using the catheter member 30 of the first configuration example, as illustrated in FIG. 8B, the catheter 100 in which the distal end tip 40 and the second member 32 forming the catheter body 10 are fused can be manufactured. Note that the cross-sectional shape and the like of the distal end tip 40 are not particularly limited. The distal end tip 40 may have, for example, a non-circular cross-sectional shape.

The method for manufacturing the catheter of the first configuration example can be applied to various catheters such as a case where a distal end tip is joined to a distal end of a guide wire lumen shaft of a balloon catheter, in addition to the catheter as illustrated in FIG. 1.

### Second Configuration Example

As illustrated in FIGS. 9A and 9B, the catheter member 30 of the second configuration example includes an inner layer and an outer layer constituting the catheter body 10, and a reinforcing body 50 disposed between the inner layer and the outer layer.

In a case where the catheter member 30 of the second configuration example is used, as illustrated in FIG. 9A, the insertion member 210 is inserted into the lumen of a second tube 12 in a state where a first tube 11 (corresponding to the “first member”) serving as an outer layer, the reinforcing body 50, and the second tube 12 (corresponding to the “second member”) serving as an inner layer are arranged in this order from the outside in the radial direction. The first tube 11 and the reinforcing body 50 together with the second tube 12 through which the insertion member 210 is moved into the first hollow portion 221 of the elastic body 220 of the manufacturing device 200 described above, so that the position is maintained. Then, the laser beam L is emitted toward the laminated portion (adjacent portion P) of the outer layer (first tube 11), the inner layer (second tube 12), and the reinforcing body 50 to form a fusion bonded portion. Together with the pressing of the elastic body 220, the fluidity of the first tube 11 is increased by the heat generated by the laser beam L emitted immediately after the elastic body 220 and the first tube 11 is brought into contact with (or adheres to) each other being absorbed by the first tube 11 and the reinforcing body 50, and the resin of the first tube 11 enters the gap of the reinforcing body 50.

As illustrated in FIG. 9B, the method for manufacturing a catheter using the catheter member 30 of the second configuration example can manufacture the catheter 100 in which the reinforcing body 50 is embedded between the inner layer and the outer layer constituting the catheter body 10. Note that the reinforcing body 50 can include, for example, a metal blade, a coil, or the like.

In FIG. 9A of the present configuration example, in order to describe a state in which the elastic body 220 applies a pressing force to the catheter member 30 (tubes 11 and 12) in more detail, a shape in which the cross-sectional shape of the elastic body 220 is deformed so as to be distorted in accordance with reality is illustrated. In other drawings such as FIG. 5C, in a case where the catheter member 30 is inside the first hollow portion 221 of the elastic body 220, the amount and shape of distortion are different due to the difference between the outer diameter of the catheter member 30 and the inner diameter of the first hollow portion 221. In the other drawings, the cross-sectional shape is circular in order to avoid complexity, but the cross-sectional shape of each elastic body 220 is also deformed to be distorted as described with reference to FIG. 9A.

### Third Configuration Example

In the catheter member 30 of the third configuration example, like an aspiration catheter, an IVUS catheter, and an atherectomy catheter, a first member 31 constituting a catheter body 10 and a guide wire shaft 60 (corresponding to a “second member”) including a guide wire lumen 61a are arranged in the radial direction.

FIG. 10A illustrates a state in which the catheter member 30 is disposed inside the first hollow portion 221 of the elastic body 220. FIG. 10B is an axis-orthogonal cross-sectional view before the first member 31 and the guide wire shaft 60 are fused, and FIG. 10C is an axis-orthogonal cross-sectional view after the first member 31 and the guide wire shaft 60 are fused.

As illustrated in FIGS. 10A and 10B, the catheter member 30 of the third configuration example is moved to the inside of the first hollow portion 221 of the elastic body 220 in a state where the members 31 and 60 are arranged in the radial direction by the guide wire shaft 60 in which the insertion member 210 is inserted into the guide wire lumen 61a on the first member 31 in which the insertion member 210 is inserted into the lumen 31a. Then, after the position is maintained, the adjacent portion P between the first member 31 and the guide wire shaft 60 is irradiated with the laser beam L to form a fusion bonded portion.

In the manufacturing method according to the third configuration example, as illustrated in FIGS. 10B and 10C, a part of the members 31 and 60 located in the adjacent portion P is melted, and the melted portions are fused to each other. During melting and curing, the members 31 and 60 receive a restraining force from the inner peripheral surface 231a of the hard component 230 disposed radially outside the members 31 and 60. Therefore, the members 31 and 60 are melted and cured in a state of receiving a compressive force in the radial direction. Therefore, in members 31 and 60, the dimension L2 in the longitudinal (long) axis direction of the cross section orthogonal to the axis after fusion illustrated in FIG. 10C is smaller than the dimension L2 in the longitudinal (long) axis direction of the cross section orthogonal to the axis before fusion illustrated in FIG. 10B.

In the method for manufacturing a catheter using the catheter member 30 of the third configuration example, as illustrated in FIG. 10C, the catheter 100 in which the first member 31 and the guide wire shaft 60 are fused can be manufactured. Note that the catheter member 30 of the third configuration example can also be used for, for example, an intermediate shaft of a balloon catheter and an inner tube shaft including a guide wire lumen.

The catheter in the method for manufacturing a catheter of the present disclosure includes, in addition to a so-called finished product that functions as a catheter after the present manufacturing, an intermediate product that becomes a finished product through a plurality of steps after the present manufacturing, and a product that becomes a part of a finished product or an intermediate product after the present manufacturing.

The detailed description above describes embodiments of a method for manufacturing a catheter. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A method for manufacturing a catheter comprising:
in a position maintaining state in which a plurality of catheter members arranged in a predetermined manner are inserted into a first hollow portion of an elastically deformable elastic body having a laser transmission property and having a first hollow portion having a cross-sectional area smaller than cross-sectional areas of fusion target portions of the plurality of catheter members at no load via an insertion member, and a position of the elastic body is maintained inside a second hollow portion of a hard component having a laser transmission property and harder than the elastic body,
irradiating the plurality of catheter members arranged in the predetermined manner via the hard component and the elastic body with a laser beam to fuse the plurality of catheter members arranged in the predetermined manner.

2. The method for manufacturing a catheter according to claim 1, wherein an inner peripheral surface of the first hollow portion of the elastic body is configured by a material in which sliding resistance with respect to the catheter member is reduced.

3. The method for manufacturing a catheter according to claim 1, wherein cross-sectional shapes of the plurality of catheter members arranged in the predetermined manner are different before and after passing through the first hollow portion of the elastic body.

4. The method for manufacturing a catheter according to claim 3, wherein cross-sectional areas of the plurality of catheter members arranged in the predetermined manner are reduced by passing through the first hollow portion of the elastic body as compared with a cross-sectional area before passing.

5. The method for manufacturing a catheter according to claim 3, wherein cross-sectional shapes of the plurality of catheter members arranged in a predetermined manner are shortened in a longitudinal axis direction by passing through the first hollow portion of the elastic body.

6. The method for manufacturing a catheter according to claim 1, wherein the plurality of catheter members arranged in the predetermined manner include a first member and a second member arranged in a radial direction.

7. The method for manufacturing a catheter according to claim 1, wherein the plurality of catheter members arranged in the predetermined manner include a first member and a second member arranged in an axial direction.

8. A method for manufacturing a catheter comprising:
in a state in which, in a state where a plurality of catheter members arranged in a predetermined manner on an insertion member positioned coaxially with a first hollow portion is disposed inside the first hollow portion of an elastically deformable elastic body having a laser transmission property and the first hollow portion through which a plurality of catheter members can be inserted at no load, and the elastic body and the plurality of catheter members arranged in the predetermined manner are disposed inside a second hollow portion of a hard component having a laser transmission property and harder than the elastic body, the plurality of catheter members arranged in the predetermined manner and the elastic body are relatively moved in a coaxial direction and a force is applied in a direction in which the hard component reduces an outer diameters of the plurality of catheter members arranged in the predetermined manner via the elastic body,
irradiating the plurality of catheter members arranged in a predetermined manner via the hard component and the elastic body with a laser beam to fuse the plurality of catheter members arranged in a predetermined manner.

9. The method for manufacturing a catheter according to claim 8, wherein an inner peripheral surface of the first hollow portion of the elastic body is configured by a material in which sliding resistance with respect to the catheter member is reduced.

10. The method for manufacturing a catheter according to claim 8, wherein cross-sectional shapes of the plurality of catheter members arranged in the predetermined manner are different before and after passing through the first hollow portion of the elastic body.

11. The method for manufacturing a catheter according to claim 10, further comprising:
reducing a cross-sectional areas of the plurality of catheter members arranged in the predetermined manner by passing through the first hollow portion of the elastic body as compared with a cross-sectional area before passing.

12. The method for manufacturing a catheter according to claim 10, wherein cross-sectional shapes of the plurality of catheter members arranged in the predetermined manner are shortened in a longitudinal axis direction by passing through the first hollow portion of the elastic body.

13. The method for manufacturing a catheter according to claim 8, wherein the plurality of catheter members arranged in the predetermined manner include a first member and a second member.

14. A method for manufacturing a catheter comprising:
inserting a plurality of catheter members via an insertion member into a first hollow portion of an elastically deformable elastic body having a laser transmission property, the first hollow portion having a cross-sectional area smaller than cross-sectional areas of fusion target portions of the plurality of catheter members;
maintaining a position of the elastic body inside a second hollow portion of a hard component having a laser transmission property and harder than the elastic body; and
irradiating the plurality of catheter members and the elastic body arranged in the predetermined manner via the hard component and the elastic body with a laser beam to fuse the plurality of catheter members arranged in the predetermined manner.

15. The method for manufacturing a catheter according to claim 14, wherein an inner peripheral surface of the first hollow portion of the elastic body is configured by a material in which sliding resistance with respect to the catheter member is reduced.

16. The method for manufacturing a catheter according to claim 14, wherein cross-sectional shapes of the plurality of catheter members arranged in the predetermined manner are different before and after passing through the first hollow portion of the elastic body.

17. The method for manufacturing a catheter according to claim 16, further comprising:
reducing a cross-sectional areas of the plurality of catheter members arranged in the predetermined manner by passing through the first hollow portion of the elastic body as compared with a cross-sectional area before passing.

18. The method for manufacturing a catheter according to claim 16, wherein cross-sectional shapes of the plurality of catheter members arranged in the predetermined manner are shortened in a longitudinal axis direction by passing through the first hollow portion of the elastic body.

19. The method for manufacturing a catheter according to claim 14, wherein the plurality of catheter members arranged in the predetermined manner include a first member and a second member arranged in a radial direction.

20. The method for manufacturing a catheter according to claim 14, wherein the plurality of catheter members arranged in a predetermined manner include a first member and a second member arranged in an axial direction.
